(19) United States
(12) Patent Application Publication
(10) Pub. No.: US 2026/0033779 A1
(43) Pub. Date: Feb. 5, 2026

(54) ELECTRONIC DEVICE AND CONTROL METHOD FOR DETERMINING USER DEHYDRATION

(71) Applicant: SAMSUNG ELECTRONICS CO., LTD., Suwon-si (KR)

(72) Inventors: Youngjae Oh, Suwon-si (KR); Hyoungseon Choi, Suwon-si (KR); Jinhong Min, Suwon-si (KR); Taehan Jeon, Suwon-si (KR); Joonho Kim, Suwon-si (KR); Taeseon Kim, Suwon-si (KR); Seoyoung Yoon, Suwon-si (KR)

(73) Assignee: SAMSUNG ELECTRONICS CO., LTD., Suwon-si (KR)

(21) Appl. No.: 19/351,709

(22) Filed: Oct. 7, 2025

Related U.S. Application Data
(63) Continuation of application No. PCT/KR2024/004329, filed on Apr. 3, 2024.

(30) Foreign Application Priority Data
Apr. 7, 2023 (KR) 10-2023-0046270

(51) Int. Cl. A61B 5/00; A61B 5/0205; A61B 5/0295; A61B 5/0531; A61B 5/0537; A63B 24/00

(52) U.S. Cl. CPC A61B 5/4875; A61B 5/0205; A61B 5/486; A61B 5/681; A61B 5/7278; A61B 5/7282; A63B 24/0062; A61B 5/0295; A61B 5/0531; A61B 5/0537

(57) ABSTRACT

An electronic device and a control method thereof are disclosed. The electronic device includes one or more sensors, an output interface, and one or more processors that: by first sensing data of the one or more sensors, identify whether a user is exercising, and when the user is exercising, provide, through the output interface and by second sensing data acquired through the one or more sensors and a first total body water analysis algorithm, information guiding a water intake recommendation before the current total body water of the user reaches a reference total body water. And when the user is not exercising, provide, through the output interface and by the second sensing data and a second total body water analysis algorithm, the information guiding the water intake recommendation when the current total body water of the user reaches the reference total body water.

### CROSS-REFERENCE TO RELATED APPLICATIONS

This application is a continuation of International Application No. PCT/KR2024/004329, filed on Apr. 3, 2024, which is based on and claims priority to Korean Patent Application No. 10-2023-0046270 filed on Apr. 7, 2023, in the Korean Intellectual Property Office, the disclosures of which are incorporated by reference herein in their entireties BACKGROUND

### 1. FIELD

The disclosure relates to an electronic apparatus and a control method for determining user dehydration, and more particularly to an electronic apparatus and a control method for determining or predicting whether there is user dehydration according to a state of a user.

### 2. DESCRIPTION OF RELATED ART

As interest related to home care is increased with recent developments in electronic technology, use of health care electronic apparatuses that obtain biometric information of a user and manage the health of the user have increased. Through the above, the user may grasp the health status of the user in real-time without having to visit a specialist and a specialized medical institution, and furthermore, receive even an analysis result of the health status of the user from the electronic apparatus.

Health care electronic apparatuses may be worn by the user, and may be implemented as wearable devices that can easily collect biometric information by contacting with the body of the user. For example, smart watches, smart glasses and the like correspond thereto. Smart watches may obtain biometric information of the user such as heart rate, temperature, and blood pressure and provide a function for analyzing the current health status of the user or analyzing a sleep pattern of the user.

Recently, with developments in sensor technology, small health care electronic apparatuses such as smart watches have also become capable of collecting a more varied biometric information about the user. Accordingly, smart watches have been able to measure a body water amount of the user based on various biometric information of the user obtained through sensors, and also provide a function of determining whether the user is dehydrated. Specifically, because excessive dehydration affects motor abilities or cognitive abilities, and can lead to death, determining whether the user is dehydrated, and managing the fluid intake of the user is key in managing the health of the user.

Further, by using an electronic device, rather than a wearable and possibly disposable patch, such as an adhesive-to-skin or elastically-contacted-to-skin patch including various microfluidic channels and chemical reagents along or in communication with those channels to react with body fluid from being adhered to the user's skin, the field and technology of monitoring user dehydration is improved by instead using the electronics as described herein.

### SUMMARY

There is provided electronic apparatus, including: one or more sensors; an output interface; and one or more processors configured to: identify, based on first sensing data obtained through the one or more sensors sensing at least any of acceleration of a user, electrical activity of a skin of the user, and a blood volume of the skin of the user, whether the user is exercising; determine, based on the user being identified as exercising and on second sensing data obtained through the one or more sensors and a first body water analysis algorithm, whether a current body water amount of the user reaches a reference body water amount; provide, through the output interface and based on the user being identified as exercising, first information guiding a fluid intake recommendation before the current body water amount of the user is determined to reach the reference body water amount; determine, based on the user being identified as not exercising and on the second sensing data and a second body water analysis algorithm that is different from the first body water analysis algorithm, whether the current body water amount of the user reaches the reference body water amount; and provide, through the output interface and based on the user being identified as not exercising, second information guiding the fluid intake recommendation when the current body water amount of the user is determined to reach the reference body water amount.

The one or more processors may be further configured to: obtain, based on the user being identified as exercising, the current body water amount of the user by applying the first body water analysis algorithm to the second sensing data obtained through the one or more sensors, and obtain, based on the user being identified as not exercising, the current body water amount of the user by applying the second body water analysis algorithm to the second sensing data obtained through the one or more sensors.

The one or more sensors may include an impedance sensor, a Photoplethysmogram (PPG) sensor, and an Electro Dermal Activity (EDA) sensor, and the second sensing data includes third sensing data, fourth sensing data, and fifth sensing data obtained respectively through the impedance sensor, the PPG sensor, and the EDA sensor such that the impedance sensor obtains the third sensing data, the PPG sensor obtains the fifth sensing data, and the EDA sensor obtains the fifth sensing data, and the one or more processors may be further configured to: obtain, based on the user being identified as exercising, a body water amount of the user by applying a first weight value, a second weight value, and a third weight value to the third sensing data, the fourth sensing data, and the fifth sensing data, respectively, and obtain, based on the user being identified as not exercising, the body water amount of the user by applying a fourth weight value, a fifth weight value, and a sixth weight value to the third sensing data, the fourth sensing data, and the fifth sensing data, respectively, and the first weight value may be same as the fourth weight value, and the second weight value may be different in sign from the fifth weight value such that a first one of the second weight value and the fifth weight value may be positive and a second one of the second weight value and the fifth weight value may be negative, and the third weight value may be different in sign from the sixth weight value.

The one or more processors may be further configured to: obtain, based on the user being identified as exercising, the second sensing data at each of a plurality of first periods through the one or more sensors; and obtain, based on the user being identified as not exercising, the second sensing data at each of a plurality of second periods through the one or more sensors, and each one of the plurality of first periods may be shorter in time than each one of the plurality of second periods.

The one or more processors may be further configured to: identify, based on the user being identified as not dehydrated, a body water change rate of the user based on a previous body water amount of the user obtained from a previous period, of any of the plurality of first periods and the plurality of second periods, and the current body water amount of the user; and provide, based on the body water change rate of the user being greater than or equal to a threshold value, information predicting that dehydration may be to occur and guiding the fluid intake recommendation to the user through the output interface.

The electronic apparatus may further include: a memory storing a plurality of first reference values, corresponding to when the user may be to be identified as exercising, and a plurality of second reference values corresponding to when the user may be to be identified as not exercising, and the one or more processors may be further configured to: compare, based on the user being identified as exercising, each of a plurality of second sensing data obtained through the one or more sensors according to the first body water analysis algorithm and the plurality of first reference values; provide, through the output interface and based on the user being identified as exercising, the first information as guiding the fluid intake recommendation before the current body water amount of the user reaches the reference body water amount based on a first comparison result; compare, based on the user being identified as not exercising, each of the plurality of second sensing data obtained through the one or more sensors according to the second body water analysis algorithm and the plurality of second reference values; and provide, through the output interface and based on the user being identified as not exercising, information guiding the fluid intake recommendation when the current body water amount of the user reaches the reference body water amount based on a second comparison result.

The one or more sensors may include an impedance sensor, a Photoplethysmogram (PPG) sensor, and an Electro Dermal Activity (EDA) sensor, and the second sensing data may include sixth sensing data, seventh sensing data, and eighth sensing data obtained respectively through the impedance sensor, the PPG sensor, and the EDA sensor such that the impedance sensor obtains the sixth sensing data, the PPG sensor obtains the seventh sensing data, and the EDA sensor obtains the eighth sensing data, and the one or more processors are further configured to: compare, based on the user being identified as exercising, a first reference value corresponding to each of the sixth sensing data, the seventh sensing data, and the eighth sensing data and obtain first change rates relative to first reference values of each thereof, and obtain the first comparison result based on the first change rates relative to first reference values of each thereof; and compare, based on the user being identified as exercising, a second reference value corresponding to each of the sixth sensing data, the seventh sensing data, and the eighth sensing data and obtain second change rates relative to second reference values of each thereof, and obtain the second comparison result based on the second change rates relative to second reference values of each thereof.

The one or more processors may be further configured to: provide, through the output interface and based on the user being identified as exercising, the first information as guiding the fluid intake recommendation before the current body water amount of the user reaches the reference body water amount and based on determining that a plurality of the first change rates, from among the first change rates, corresponding to each of the sixth sensing data, the seventh sensing data, and the eighth sensing data satisfies a pre-set first change condition, and provide, through the output interface and based on the user being identified as not exercising, the second information as guiding the fluid intake recommendation when the current body water amount of the user may be determined to reach the reference body water amount and based on a plurality of the second change rates, from among the second change rates, corresponding to each of the sixth sensing data, the seventh sensing data, and the eighth sensing data satisfies a pre-set second change condition.

The one or more processors may be further configured to: obtain, based on the user being identified as exercising, biometric data of the user through the one or more sensors for a pre-set time; and re-identify whether the user may be exercising by comparing the biometric data and with reference biometric data of the user, and the biometric data of the user may include one or more of a heart rate, a temperature, and skin electrical signals.

The one or more processors may be further configured to: output, through the output interface and based on the user being identified both as exercising and as dehydrated, first guide information including at least one from among a first message, recommending to the user of adjusting an exercise intensity, and a second message recommending an intake of a beverage; and output, through the output interface and based on the user being identified both as not exercising and as dehydrated, second guide information including at least one from among a third message, warning of a danger of chronic dehydration to the user, and a fourth message recommending to improve living habits of the user.

The electronic apparatus may be a smart watch configured to be worn by the user, the smart watch may be configured to, while worn by the user, automatically and in real-time: control the one or more sensors to obtain the first sensing data; determine, based on the first sensing data, whether the user may be exercising; determine, based on the user being identified as exercising and on the second sensing data and the first body water analysis algorithm, whether the current body water amount of the user reaches the reference body water amount; provide, through the output interface and based on the user being identified as not exercising, the first information, determine, based on the user being identified as not exercising and on the second sensing data and the second body water analysis algorithm that may be different from the first body water analysis algorithm, whether the current body water amount of the user reaches the reference body water amount; and providing, through the output interface and based on the user being identified as not exercising, the second information.

There is provided a method for controlling an electronic apparatus, the method, by one or more processors of the electronic apparatus, including: identifying, based on first sensing data obtained through one or more sensors of the electronic apparatus sensing at least any of acceleration of a user, electrical activity of a skin of the user, and a blood volume of the skin of the user, whether the user is exercising; determining, based on the user being identified as exercising and on second sensing data obtained through the one or more sensors and a first body water analysis algorithm, whether a current body water amount of the user reaches a reference body water amount; providing, through an output interface of the electronic apparatus and based on the user being identified as exercising, first information guiding a fluid intake recommendation before the current body water amount of the user is determined to reach the reference body water amount; determining, based on the user being identified as not exercising and on the second sensing data and a second body water analysis algorithm that is different from the first body water analysis algorithm; and providing, through the output interface and based on the user being identified as not exercising, second information guiding the fluid intake recommendation when the current body water amount of the user is determined to reach the reference body water amount.

The providing first guide information may include obtaining, based on the user being identified as exercising, the current body water amount of the user by applying the first body water analysis algorithm to the second sensing data obtained through one or more sensors, and the providing second guide information may include obtaining, based on the user being identified as not exercising, the current body water amount of the user by applying the second body water analysis algorithm to the second sensing data obtained through the one or more sensors.

The one or more sensors may include an impedance sensor, a Photoplethysmogram (PPG) sensor, and an Electro Dermal Activity (EDA) sensor, and the second sensing data may include third sensing data, fourth sensing data, and fifth sensing data obtained respectively through the impedance sensor, the PPG sensor, and the EDA sensor such that the impedance sensor obtains the third sensing data, the PPG sensor obtains the fifth sensing data, and the EDA sensor obtains the fifth sensing data, and the providing first guide information may include obtaining, based on the user being identified as exercising, a body water amount of the user by applying a first weight value, a second weight value, and a third weight value to the third sensing data, the fourth sensing data, and the fifth sensing data, respectively, and the providing second guide information may include obtaining, based on the user being identified as not exercising, the body water amount of the user by applying a fourth weight value, a fifth weight value, and a sixth weight value to the third sensing data, the fourth sensing data, and the fifth sensing data, respectively, and the first weight value may be same as the fourth weight value, and the second weight value may be different in sign from the fifth weight value such that a first one of the second weight value and the fifth weight value may be positive and a second one of the second weight value and the fifth weight value may be negative, and the third weight value may be different in sign from the sixth weight value.

The providing first guide information may include obtaining, based on the user being identified as exercising, the second sensing data at each of a plurality of first periods through the one or more sensors, and the providing second guide information may include obtaining, based on the user being identified as not exercising, the second sensing data at each of a plurality of second periods through the one or more sensors, and the each one of the plurality of first periods may be shorter in time than each one of the plurality of second periods.

There is provided a non-transitory computer-readable recording medium storing computer instructions for an electronic apparatus to perform operations that, when executed by one or more processors of the electronic apparatus, cause the electronic apparatus to implement: identifying, based on first sensing data obtained through one or more sensors of the electronic apparatus sensing at least any of acceleration of a user, electrical activity of a skin of the user, and a blood volume of the skin of the user, whether the user is exercising; determining, based on the user being identified as exercising and on second sensing data obtained through the one or more sensors and a first body water analysis algorithm, whether a current body water amount of the user reaches a reference body water amount; providing, through an output interface of the electronic apparatus and based on the user being identified as exercising, first information guiding a fluid intake recommendation before the current body water amount of the user is determined to reach the reference body water amount; determining, based on the user being identified as not exercising and on the second sensing data and a second body water analysis algorithm that is different from the first body water analysis algorithm; and providing, through the output interface and based on the user being identified as not exercising, second information guiding the fluid intake recommendation when the current body water amount of the user is determined to reach the reference body water amount.

### BRIEF DESCRIPTION OF THE DRAWINGS

The above and other aspects, features, and advantages of specific embodiments of the present disclosure will be more apparent from the following description with reference to the accompanying drawings, in which:

FIG. 1 is a diagram illustrating an example of an electronic apparatus determining whether a user is dehydrated according to one or more embodiments of the disclosure;

FIG. 2 is a diagram illustrating a configuration of an electronic apparatus according to one or more embodiments of the disclosure;

FIG. 3 is a flowchart illustrating a method for controlling an electronic apparatus according to one or more embodiments of the disclosure;

FIG. 4 is a flowchart illustrating a method for controlling an electronic apparatus to re-identify whether a user is exercising according to one or more embodiments of the disclosure;

FIG. 5 is a diagram illustrating an example of differently applying a body water analysis algorithm according to a state of a user according to one or more embodiments of the disclosure;

FIG. 6A and FIG. 6B are diagrams each illustrating changes in sensing data obtained by an EDA sensor based on user dehydration while a user is exercising and while the user is not exercising according to one or more embodiments of the disclosure;

FIG. 7 is a diagram illustrating an example of outputting guide information recommending fluid intake to a user who is exercising and a user who is not exercising at different time-points according to one or more embodiments of the disclosure; and

FIG. 8 is a diagram illustrating a detailed configuration of an electronic apparatus according to one or more embodiments of the disclosure.

### DETAILED DESCRIPTION

Terms used in the disclosure will be briefly described, and the disclosure will be described in detail.

Terms used in describing embodiments of the disclosure are general terms selected that are currently widely used considering their function herein. However, the terms may change depending on intention, legal or technical interpretation, emergence of new technologies, and the like of those skilled in the related art. Further, in certain cases, there may be terms arbitrarily selected, and in this case, the meaning of the term will be disclosed in greater detail in the corresponding description. Accordingly, the terms used herein are not to be understood simply as its designation but based on the meaning of the term and the overall context of the disclosure.

In the disclosure, expressions such as “have”, “may have”, “include”, and “may include” are used to designate a presence of a relevant characteristic (e.g., elements such as numerical value, function, operation, or component), and not to preclude a presence or a possibility of additional characteristics.

In the disclosure, expressions such as “A or B”, “at least one of A and/or B”, or “one or more of A and/or B” may include all possible combinations of the items listed together. For example, “A or B”, “at least one of A and B”, or “at least one of A or B” may refer to all cases including (1) at least one A, (2) at least one B, or (3) both of at least one A and at least one B.

Expressions such as “1st”, “2nd”, “first”, or “second” used in the disclosure may limit various elements regardless of order and/or importance, and may be used merely to distinguish one element from another element and not limit the relevant element.

When a certain element (e.g., a first element) is indicated as being “(operatively or communicatively) coupled with/to” or “connected to” another element (e.g., a second element), it may be understood as the certain element being directly coupled with/to the another element or as being coupled through other element (e.g., a third element).

The expression “configured to . . . (or set up to)” used in the disclosure may be used interchangeably with, for example, “suitable for . . . ”, “having the capacity to . . . ”, “designed to . . . “, “adapted to . . . “, “made to . . . “, or “capable of . . . ” based on circumstance. The term “configured to . . . (or set up to)” may not necessarily mean “specifically designed to” in terms of hardware.

In a certain circumstance, the expression “a device configured to . . . ” may mean something that the device “may perform . . . ” together with another device or components. For example, a phrase “a processor configured to (or set up to) perform A, B, or C” may mean a dedicated processor for performing a relevant operation (e.g., an embedded processor), or a generic-purpose processor (e.g., a central processing unit (CPU) or an application processor) capable of performing the relevant operations by executing one or more software programs stored in a memory device.

A singular expression includes a plural expression, unless otherwise specified. It is to be understood that the terms such as “configured” or “include” are used herein to designate a presence of a characteristic, number, step, operation, element, component, or a combination thereof, and not to preclude a presence or a possibility of adding one or more of other characteristics, numbers, steps, operations, elements, components or a combination thereof.

The term “module” or “part” used in the embodiments perform at least one function or operation, and may be implemented with hardware or software, or implemented with a combination of hardware and software. In addition, a plurality of “modules” or a plurality of “parts”, except for a “module” or a “part” which needs to be implemented with a specific hardware, may be integrated in at least one module and implemented as at least one processor.

Meanwhile, various elements and areas of the drawings have been schematically illustrated. Accordingly, the technical spirit of the disclosure is not limited by relative sizes and distances illustrated in the accompanied drawings.

An embodiment of the disclosure will be described in greater detail below with reference to the accompanied drawings.

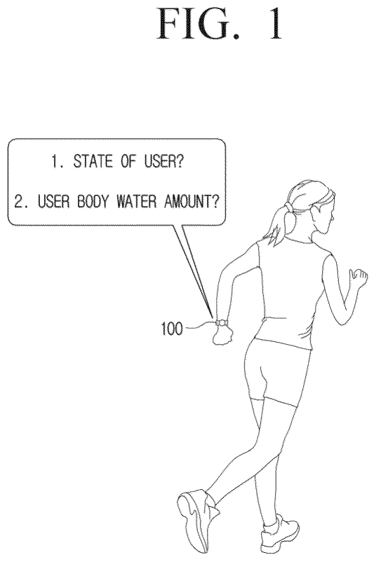

FIG. 1 is a diagram illustrating an example of an electronic apparatus determining whether a user is dehydrated according to one or more embodiments of the disclosure.

An electronic apparatus 100 according to one or more embodiments of the disclosure may obtain various biometric information of the user through one or more sensors. In an example, the electronic apparatus 100 may be attached to one portion of the body of the user and obtain biometric information such as heart rate, temperature, and blood pressure of the user. Further, the electronic apparatus 100 may identify a body water amount of the user based on the obtained biometric information, and determine whether the user is dehydrated based on the identified body water amount.

However, biometric information of the user obtained by the electronic apparatus 100 may be different such as a size of sensing value and a change rate even if it is sensing data obtained by a same sensor (i.e., a second sensor) according to a state of the user. For example, sensing data such as heart rate, temperature, and blood pressure obtained from a user who is exercising may be different from sensing data from a user who is sleeping. Furthermore, a change rate of body water may be different according to a state of a user. For example, even if a same body water amount is identified in a user who is exercising and in a user who is sleeping, body water loss of the user who is exercising may be relatively faster. As described, because of different information implications according to a state of a user even if it is sensing data obtained through the same sensor, the electronic apparatus 100 of the disclosure may classify body water analysis algorithms for application according to a state of a user. That is, the electronic apparatus 100 may identify a body water amount of a user or changes in body water by identifying a state of a user and then analyzing sensing data obtained by using a body water analysis algorithm corresponding to the identified user state. And so, not only is the field of sensing dehydration of a user improved by embodiments herein by using an electronic device as compared to adhesive patches with microfluidic channels and chemical reagents or the like, but the use by electronic device itself is also improved by embodiments herein by its discriminating between such states of the user.

In addition thereto, the electronic apparatus 100 of the disclosure may distinguish an output time-point of guide information that warns the user of dehydration symptoms to occur according to the state of the user. For example, when dehydration rapidly progresses as if during exercise, information warning the user of dehydration symptoms may be output before dehydration symptoms occur. In other words, because a degree of risk of dehydration symptoms and time until recovering from dehydration symptoms may be different according to the state of the user, the electronic apparatus 100 may guide the user to more appropriately manage their health from dehydration symptoms by outputting guide information at an appropriate time-point according to the state of the user.

One or more embodiments of the disclosure associated with the above will be described below.

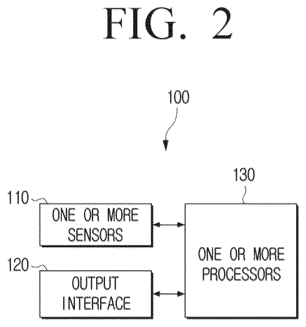

FIG. 2 is a diagram illustrating a configuration of the electronic apparatus 100 according to one or more embodiments of the disclosure.

Referring to FIG. 2, the electronic apparatus 100 may include one or more sensors 110, an output interface 120, and one or more processors 130.

The electronic apparatus 100 may include a plurality of sensors, the output interface 120, and one or more processors 130, and may be implemented as a wearable electronic apparatus 100 in a form that can be worn by the user or attached or inserted in the skin. In an example, the electronic apparatus 100 may be a smart watch, a smart band, a smart glass, a smart ring, a head mounted display (HMD), and the like.

However, the embodiment is not limited thereto, and may be any apparatus so long as it is an electronic apparatus 100 implemented in a form that can be worn by the user or attached or inserted in the skin. For example, the above may include a band, an adhesive portion, and the like and may be implemented as a smart phone, a tablet PC, and the like capable of being worn by or attached to the user. For convenience of description of the disclosure, it will be described below assuming that the electronic apparatus 100 is a smart watch.

The one or more sensors 110 may include a plurality of sensors of various types. The one or more sensors 110 may measure a physical amount or detect an operating state of the electronic apparatus 100, and convert the measured or detected information into electrical signals. The sensors 110 may include a camera, and the camera may include a lens which focuses visible light and other optical signals received by being reflected by an object to an image sensor and an image sensor capable of detecting visible light and other optical signals. Here, the image sensor may include a 2D pixel array divided into a plurality of pixels.

The one or more sensors 110 according to one or more embodiments may include at least one from among an impedance sensor, a Photoplethysmograpy (PPG) sensor, an Electrodermal activity (EDA) sensor, a temperature sensor, or an acceleration sensor. The PPG sensor may be a sensor that checks heartbeat activity state by measuring an amount of blood flow flowing in the veins by using optical properties of the skin. The EDA sensor may be a sensor that provides information about the autonomic nervous system of a human body by combining with the heart rate, number of breaths, blood pressure, and the like of a human. A processor 130 may use a pre-set algorithm, and obtain body water data of the user through sensing information received from at least one from among the impedance sensor, the Photoplethysmograpy (PPG) sensor, the Electrodermal activity (EDA) sensor, or the temperature sensor.

The one or more sensors 110 may obtain various information about the user. Here, various information may include direct biometric information such as the heart rate, blood pressure, temperature, and the like of the user. Alternatively, in addition to the direct biometric information such as impedance information with respect to a user determined based on electrical signals transferred to the user by one sensor, various information obtainable from the user that is used to determine the biometric information of the user (e.g., body water amount of the user, muscle mass, etc.) may be included.

Alternatively, various information may include information used in identifying an exercise state of the user such as acceleration information, angular speed information, and the like. To this end, the one or more sensors 110 may be implemented with an angular speed sensor, an acceleration sensor, a gyro sensor, an IMU sensor, and the like.

For convenience of description below, the one or more sensors 110 will be divided into a first sensor and a second sensor according to the type of information obtained through the sensors. Specifically, the one or more sensors 110 used in identifying the exercise state of the user may be designated as the first sensor, and the one or more sensors 110 used in obtaining biometric information and data obtained to determine the biometric information may be designated as the second sensor. Further, sensing data obtained by the first and second sensors may be designated as first sensing data and second sensing data, respectively.

The output interface 120 may be a device that outputs information of various types in order to provide the user with information. According to one or more embodiments, the output interface 120 may be implemented as at least one of a speaker or a display, but is not limited thereto.

The output interface 120 may output guide information recommending fluid intake to the user in case dehydration symptoms occur to the user or in case dehydration symptoms are predicted to occur. In an example, the output interface 120 may visually output a message recommending fluid intake to the user through the display, and convert a message recommending fluid intake to the user to voice signals and output through the speaker.

Specifically, the output interface 120 may output guide information that is distinguished according to the state of the user. In other words, the output interface 120 may differently output guide information according to the state of the user for the user who is experiencing dehydration symptoms to take appropriate measures according to the state of the user.

The one or more processors 130 may control the overall operation and function of the electronic apparatus 100 by being electrically connected with the one or more sensors 110 and the output interface 120.

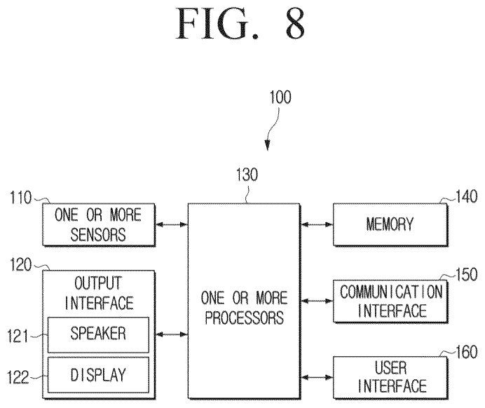

The one or more processors 130 may include one or more from among a central processing unit (CPU), a graphics processing unit (GPU), an accelerated processing unit (APU), a many integrated core (MIC), a digital signal processor (DSP), a neural processing unit (NPU), a hardware accelerator, or a machine learning accelerator. The one or more processors 130 may control one or a random combination from among other elements of the electronic apparatus 100, and perform an operation associated with communication or data processing. The one or more processors 130 may execute one or more programs or instructions stored in the memory. For example, the one or more processors 130 may perform, by executing one or more instructions stored in a memory 140 (see FIG. 8 for example), a method according to one or more embodiments of the disclosure.

When a method according to one or more embodiments of the disclosure includes a plurality of operations, the plurality of operations may be performed by one processor of the one or more processors 130, or performed by a plurality of processors of the one or more processors 130. For example, when a first operation, a second operation, and a third operation are performed by a method according to one or more embodiments, the first operation, the second operation, and the third operation may all be performed by a first processor, or the first operation and the second operation may be performed by the first processor (e.g., a generic-purpose processor) and the third operation may be performed by a second processor (e.g., an artificial intelligence dedicated processor).

The one or more processors 130 may be implemented as a single core processor that includes one core, or as one or more multicore processors that include a plurality of cores (e.g., a homogeneous multicore or a heterogeneous multicore). If the one or more processors 130 are implemented as the multicore processors, each of the plurality of cores included in the multicore processors may include memory inside the processor such as a cache memory and an on-chip memory, and a common cache shared by the plurality of cores may be included in the multicore processors. In addition, each of the plurality of cores (or a portion from among the plurality of cores) included in the multicore processors may independently read and perform a program command for implementing a method according to one or more embodiments of the disclosure, or read and perform a program command for implementing a method according to one or more embodiments of the disclosure due to a whole (or a portion) of the plurality of cores being interconnected.

When a method according to one or more embodiments of the disclosure includes a plurality of operations, the plurality of operations may be performed by one core from among the plurality of cores or performed by the plurality of cores included in the multicore processors. For example, when a first operation, a second operation, and a third operation are performed by a method according to one or more embodiments, the first operation, the second operation, and the third operation may all be performed by a first core included in the multicore processors, or the first operation and the second operation may be performed by the first core included in the multicore processors and the third operation may be performed by a second core included in the multicore processors.

In an embodiment of the disclosure, the one or more processors 130 may refer to a system on chip (SoC), the single core processor, or the multicore processors in which the one or more processors 130 and other electronic components are integrated or a core included in the single core processor or the multicore processor, and the core herein may be implemented as the CPU, the GPU, the APU, the MIC, the DSP, the NPU, the hardware accelerator, the machine learning accelerator, or the like, but the embodiments of the disclosure are not limited thereto.

For convenience of description, the one or more processors 130 will be referred to as the processor 130 below.

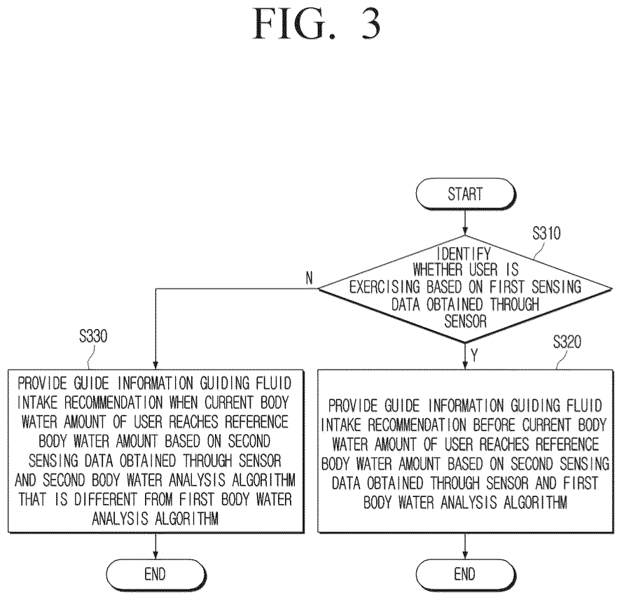

FIG. 3 is a flowchart illustrating a method for controlling the electronic apparatus 100 according to one or more embodiments of the disclosure.

Referring to FIG. 3, according to one or more embodiments of the disclosure, the processor 130 may identify a state of the user based on sensing data (hereinafter, referred to as ‘first sensing data’) obtained through one or more first sensors (S310). Specifically, the processor 130 may identify various states of the user such as, for example, and without limitation, whether the user is exercising, whether the user is eating, whether the user is sleeping, and the like based on first sensing data obtained through the one or more first sensors. Because size, change rate, and the like of sensing values obtained by the same sensor (i.e., second sensor) can be different according to the state of the user, this is to more accurately determine the body water of the user by taking into consideration the state of the user. In other words, the processor 130 may first determine which type the state of the user belongs based on the first sensing data in order to apply an appropriate analysis algorithm according to the state of the user.

However, for convenience of description of the disclosure below, the state of the user will be described assuming that the user is exercising and the user is not exercising.

In an example, the processor 130 may identify whether the user is exercising by using at least one from among a plurality of first sensors. Specifically, the processor 130 may obtain acceleration data associated with the user using the acceleration sensor, or obtain angular speed data associated with the user using the angular speed sensor. Alternatively, the acceleration data, the angular speed data, and geomagnetic data associated with the user may be simultaneously obtained by using the IMU sensor. Specifically, the processor 130 may obtain various first sensing data associated with the user by simultaneously using the above-described acceleration sensor, the angular speed sensor, and the IMU sensor.

At this time, the processor 130 may identify whether the user is exercising by comparing the obtained first sensing data with a reference value. In an example, the processor 130 may identify, based on an acceleration value of the user included in the acceleration data being greater than or equal to a pre-set acceleration value, the user as exercising. Alternatively, the processor 130 may identify, based on an angular speed value of the user included in the angular speed data being greater than or equal to a pre-set angular speed value, the user as exercising. Here, the reference value for determining whether the user is exercising may be sensing data obtained through the first sensor while the user is not exercising.

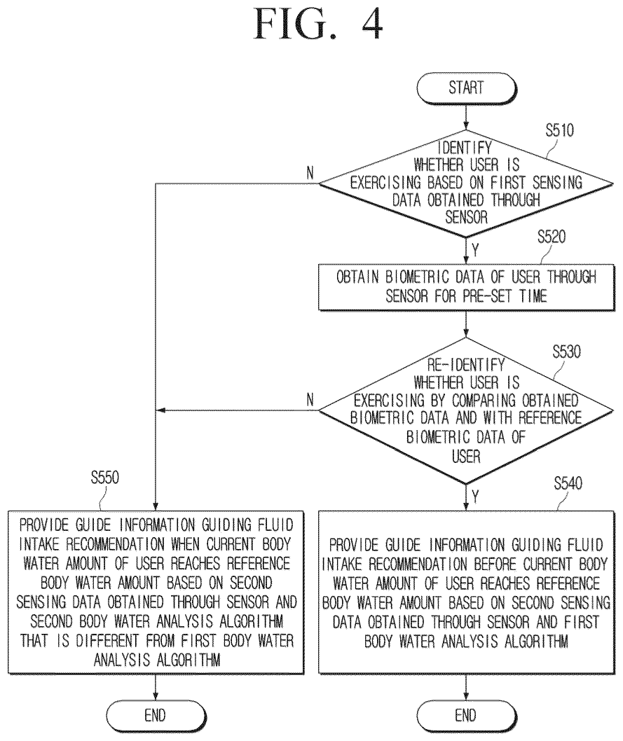

FIG. 4 is a flowchart illustrating a method for controlling the electronic apparatus 100 to re-identify whether a user is exercising according to one or more embodiments of the disclosure. Meanwhile, steps S510, S540, and S550 shown in FIG. 4 correspond respective to steps S310, S320, and S330 shown in FIG. 3.

The processor 130 may re-identify, based on the user being identified as exercising based on the first sensing data, whether the user is exercising using the second sensor. In other words, the processor 130 may perform a process of re-identifying whether the user is exercising based on a sensor of a different type (i.e., second sensor) that is not the first sensor and sensing data (i.e., second sensing data) obtained through the sensor of a different type. Through the above, the processor 130 may identify the state of the user more accurately.

In this respect, referring to FIG. 4, the processor 130 according to one or more embodiments of the disclosure may obtain, based on the user being identified as exercising based on the first sensing data, biometric data of the user through the one or more sensors 110 for a pre-set time (S520). Here, the biometric data of the user may include heart rate, temperature, and skin electrical signals.

The processor 130 may obtain biometric data (i.e., second sensing data) of the user through the second sensor. Specifically, the processor 130 may obtain impedance data about the user using at least one from among an impedance sensor, a Photoplethysmogram (PPG) sensor, and Electro Dermal Activity (EDA) sensor.

Then, the processor 130 may re-identify whether the user is exercising by comparing the obtained biometric data and with reference biometric data of the user (S530). Specifically, the processor 130 may determine, based on the heart rate included in the obtained biometric data being greater than or equal to a pre-set heart rate, the user as exercising. Alternatively, the processor 130 may determine, based on the temperature included in the obtained biometric data being greater than or equal to a pre-set temperature, the user as exercising. Alternatively, the processor 130 may determine, based on the impedance with respect to the user included in the obtained biometric data exceeding a pre-set margin of error from a pre-set impedance, the user as exercising.

Meanwhile, in order to re-identify whether the user is exercising based on the obtained biometric data, the processor 130 may perform a process of obtaining reference data about the user. In an example, the processor 130 may output a message requesting to the user to maintain a state of not exercising through the output interface 120, and obtain biometric data of the user who is not exercising through the one or more sensors 110. Then, the processor 130 may set reference values (reference heart rate, reference temperature, reference impedance, etc.) for re-identifying whether the user is exercising based on the obtained biometric data. Then, the processor 130 may store the set reference values (reference heart rate, reference temperature, reference impedance, etc.) in the memory (not shown) after setting the reference values (reference heart rate, reference temperature, reference impedance, etc.).

As described, the processor 130 may identify, based on a sensor of a different type from each other (i.e., first and second sensors) and a sensing data of a different type from each other (i.e., first and second sensing data), whether the user is exercising, identify more accurately the state of the user, and apply a body water analysis algorithm suitable to the state of the user.

Referring back to FIG. 3, the processor 130 may provide, based on the user being identified as exercising, information guiding a fluid intake recommendation before a current body water amount of the user reaches a reference body water amount through the output interface 120 based on the second sensing data obtained through the one or more sensors 110 and a first body water analysis algorithm (S320).

Then, the processor 130 may provide, based on the user being identified as not exercising, information guiding a fluid intake recommendation when the current body water amount of the user reaches the reference body water amount through the output interface 120 based on the second sensing data and a second body water analysis algorithm that is different from the first body water analysis algorithm (S330).

Specifically, the processor 130 may obtain, based on the user being identified as exercising based on the first sensing data obtained through the first sensor, or the user being re-identified as exercising based on the second sensing data obtained through the second sensor after having identified the user as exercising based on the first sensing data obtained through the first sensor, sensing data for determining whether the user is dehydrated through the second sensor.

Here, the sensing data for determining whether the user is dehydrated may be similar with the biometric data of the user (i.e., second sensing data) in that it is obtained through the second sensor, but may be distinguished in that a target for determination (i.e., whether or not dehydrated and whether or not exercising) and a method of use are different, respectively.

At this time, the processor 130 may apply a period for obtaining the second sensing data differently at each state of the user (i.e., a state in which the user is exercising and a state in which the user is not exercising). In an example, the processor 130 may obtain, based on the user being identified as exercising, the second sensing data for every first period through the one or more sensors 110. Then, the processor 130 may obtain, based on the user being identified as not exercising, the second sensing data for every second period through the one or more sensors 110. At this time, the first period may be set to a shorter time than the second period.

In other words, the processor 130 may determine, when the user is exercising, a current body water amount of the user by obtaining the second sensing data more frequently. If the user is exercising, because body water loss of the user can progress quickly than when the user is not exercising, a body water amount of the user may be identified at a shorter period (i.e., frequently) than when the user is not exercising.

In addition, the processor 130 may obtain the second sensing data about the user periodically using one or more second sensors at every third period set to a time that is longer than the first and second periods, and obtain the second sensing data by applying the first and second periods according to the state of the user (i.e., whether the user is exercising or not exercising) after identifying the state of the user when the body water amount of the user is detected as reduced based on the obtained second sensing data.

For example, if the first period is set as ten minutes, the second period is set as thirty minutes, and the third period is set as one hour, the processor 130 may obtain the second sensing data at every hour (third period). At this time, the processor 130 may obtain, based on the body water amount of the user being detected as reduced based on the second sensing data obtained at every hour (third period), the first sensing data about the user through the one or more first sensors to identify the state of the user. Then, the processor 130 may identify whether the user is exercising or not exercising based on the obtained first sensing data. If the user is identified as exercising, the processor 130 may identify the body water amount or a change in body water of the user by obtaining the second sensing data at every ten minutes (first period), and if the user is identified as not exercising, the body water amount or the change in body water of the user may be identified by obtaining the second sending data at every thirty minutes (second period).

Meanwhile, according to one or more embodiments of the disclosure, the one or more second sensors may include the impedance sensor, the PPG sensor, and the EDA sensor. At this time, the processor 130 may obtain sensing data about the user through a plurality of second sensors (impedance sensor, PPG sensor, and EDA sensor), respectively.

Specifically, the processor 130 may obtain an impedance value (or a size value of current (or voltage) received by the impedance sensor) with respect to the user through the impedance sensor. In addition, the processor 130 may obtain a strength value (or an amount of blood flow value corresponding to a strength value of an optical signal) of an optical signal (or PPG signal) obtained through the PPG sensor. In addition, the processor 130 may obtain a size value of the skin electrical signal of the user (or an impedance value the skin of the user corresponding to the skin electrical signal) through the EDA sensor.

For convenience of description of the disclosure, a sensing value obtained by the impedance sensor may be referred to as third sensing data, sensing data obtained through the PPG sensor may be referred to as fourth sensing data, and sensing data obtained through the EDA sensor may be referred to as fifth sensing data below.

The processor 130 may determine, based on the third sensing data, the fourth sensing data, and the fifth sensing data obtained respectively through the impedance sensor, the PPG sensor, and the EDA sensor, whether a current body water amount of the user who is exercising has not yet reached the reference body water amount or whether a current body water amount of the user who is not exercising has reached the reference body water amount. Here, the reference body water amount may be a body water amount that can generate dehydration symptoms in the user. In an example, the reference body water amount may be a value identified as body water of 2% body weight of the user being lost.

The current body water amount being identified as having reached the reference body water amount may be the current body water amount matching with the reference body water amount or the current body water amount being less than or equal to the reference body water amount.

In addition, the current body water amount not having reached the reference body water amount may be the current body water amount being within a pre-set range (or percentage) from the reference body water amount. In other words, even if the current body water amount has not reached the value identified as body water of 2% body weight of the user being lost, the processor 130 may identify the current body water amount as not having reached the reference body water amount yet so long as it falls within a certain percentage range (e.g., reference body water amount×110%≥current body water amount>reference body water amount) of the value that is identified as body water of 2% body weight being lost. Alternatively, the processor 130 may identify whether the current body water amount has not yet reached the reference body water amount by applying a different reference value from the reference body water amount. For example, if the reference body water amount is the value identified as the body water of 2% body weight of the user being lost, the processor 130 may set a value identified as body water of 1.8% of body weight of the user being lost as a reserve reference body water amount, and identify the current body water amount as not having reached the reference body water amount if the current body water amount of the user has reached the reserve reference body water amount.

For convenience of description of the disclosure, it will be described below assuming that the processor 130 identifies whether the current body water amount has not yet reached the reference body water amount by setting the reserve reference body water amount.

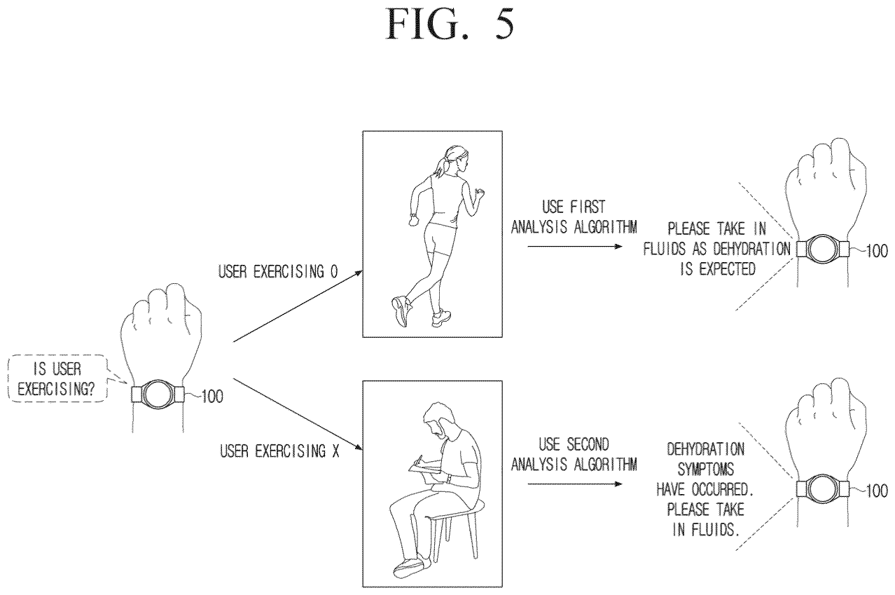

FIG. 5 is a diagram illustrating an example of differently applying a body water analysis algorithm according to a state of a user according to one or more embodiments of the disclosure.

Referring to FIG. 5, the processor 130 according to one or more embodiments of the disclosure may identify whether a current body water amount of the user has not yet reached a reference body water amount or whether the current body water amount of the user has reached the reference body water amount in each state of the user (i.e., state in which the user is exercising and state in which the user is not exercising) by using different body water analysis algorithms from each other.

Specifically, the processor 130 may identify whether the current body water amount of the user has not yet reached the reference body water amount by applying the second sensing data and the first body water analysis algorithm while the user is exercising. Conversely, the processor 130 may identify whether the current body water amount of the user has reached the reference body water amount by applying the second sensing data and the first body water analysis algorithm while the user is exercising.

An embodiment of the disclosure in which user dehydration symptoms are predicted or determined by obtaining the body water amount will be described in detail below.

First, the processor 130 may obtain, based on the user being identified as exercising, the current body water amount of the user by applying the first body water analysis algorithm to the second sensing data obtained through the one or more sensors 110. Then, the processor 130 may obtain, based on the user being identified as not exercising, the current body water amount of the user by applying the second body water analysis algorithm to the second sensing data obtained through the one or more sensors 110.

In other words, the processor 130 may identify the current body water amount of the user in each state (state in which the user is exercising and state in which the user is not exercising) based on the body water analysis algorithms (first and second analysis algorithms) that are different from each other. Then, the processor 130 may identify, by comparing the identified current body water amount of the user with the reference body water amount, whether the current body water amount of the user has reached the reference body water amount or not yet reached a pre-set body water amount.

To this end, the processor 130 may set the reference body water amount which is a subject of comparison of the current body water amount. Specifically, the processor 130 may periodically obtain the second sensing data about the user through the second sensor (e.g., for every third period), and identify the body water amount of the user. At this time, the processor 130 may set, based on previous body water amounts of a plurality of identified users from a plurality of previous periods, a reference body water amount by determining a value identified as body water of 2% body weight of the user being lost at the current period. For example, if the processor 130 identified the current body water amount of the user based on the second sensing data obtained through the second sensor at a t5 time-point, the processor 130 may set the reference body water amount for determining whether there are user dehydration symptoms currently based on the body water amounts of the user identified at each of t1 to t4 time-points.

At this time, the processor 130 may identify, based on the previous body water amounts of the plurality of identified users at the plurality of previous periods (e.g., t1 to t4), an average body water amount of the user, and determine a user body water amount at which the body water corresponding to 2% body weight of the user is lost based on the identified average body water amount. For example, assuming that the body weight of the user is 80 kg, and the average body water amount of the user is 56 kg, the processor 130 may determine 54.4 kg (56 kg-80 kg×2%) as the reference body water amount.

Alternatively, the processor 130 may identify, based on the previous body water amounts of the plurality of identified users at the plurality of previous periods (e.g., t1 to t4), a time-point at which the body water amount of the user begins to reduce, and set the body water amount of the user at the time-point at which the body water amount of the user began to reduce as the reference body water amount.

Meanwhile, the processor 130 may identify, when the body water amount of a currently identified user reaches the reference body water amount, as dehydration symptoms having occurred to the user. Referring to the above-described example, the processor 130 may identify as dehydration symptoms having occurred to the user if the current body water amount of the user reaches 54.4 kg. At this time, the processor 130 may identify, based on the current body water amount of the user being less than or equal to 54.4 kg, as the current body water amount having reached the reference body water amount.

However, the processor 130 may provide, if the user is exercising, guide information recommending fluid intake to the user by determining whether the current body water amount of the user has not yet reached the reference body water amount. In other words, if the user is exercising, the processor 130 may provide guide information recommending fluid intake to the user before dehydration symptoms occur to the user.

If the user is exercising, body water loss of the user may progress quickly different to when the user is not exercising. Accordingly, if the user is exercising, the processor 130 may recommend fluid intake to the user before the current body water amount of the user even reaches the reference body water amount (i.e., before dehydration symptoms occur to the user).

To this end, another reference body water amount (i.e., reserve reference body water amount) may be applied together unlike when the user is not exercising. For example, the processor 130 may identify, based on the user being identified as exercising, whether there is loss by an amount corresponding to 1.8% of the body weight of the user before the body water of the user loses an amount corresponding to 2% of the body weight of the user, and provide, based on the body water amount of the user being identified as having lost by an amount corresponding to 1.8% of the body weight of the user, information guiding a fluid intake recommendation through the output interface 120. Referring back to the above-described example, if the body weight of the user is 80 kg, and the reference body water amount is 54.4 kg with a body water loss of 1.6 kg, the processor 130 may provide, based on the body water amount of the user who is not exercising reaching 54.4 kg, information recommending fluid intake to the user through the output interface 120.

However, the processor may provide, based on the user exercising, information recommending fluid intake to the user through the output interface 120 when the current body water amount of the user reaches a value of 54.56 kg (a value identified as body water of 1.8% body weight of the user as being lost) and not a value of 54.4 kg (a value identified as body water of 2% body weight of the user as being lost).

According to one or more embodiments of the disclosure, the processor 130 may obtain, based on the user being identified as exercising, the body water amount of the user by applying a first weight value, a second weight value, and a third weight value to the third sensing data, the fourth sensing data, and the fifth sensing data, respectively. Then, the processor 130 may obtain, based on the user being identified as not exercising, the body water amount of the user by applying a fourth weight value, a fifth weight value, and a sixth weight value to the third sensing data, the fourth sensing data, and the fifth sensing data, respectively. At this time, the first weight value may be same as the fourth weight value, the second weight value may be different in sign with the fifth weight value, and the third weight value may be different in sign with the sixth weight value.

Specifically, the processor 130 may obtain the current body water amount of the user who is exercising or the current body water amount of the user who is not exercising based on third sensing data obtained through the impedance sensor and fourth sensing data obtained through the PPG sensor and fifth sensing data obtained through the EDA sensor.

The processor 130 may obtain, based on the user exercising, the current body water amount of the user based on Equation 1 below. Here, a may represent the first weight value, b may represent the second weight value, and c may represent the third weight value. Further, n1 may represent a first coefficient, n2 may represent a second coefficient, and n3 may represent a third coefficient:

$$Body water amount of use who is exercising = a \times third sensing {data}^{n 1} + b \times fourth sensing {data}^{n 2} + c \times fifth sensing {data}^{n 3} \qquad ( Equation 1 )$$

In addition, the processor 130 may obtain, based on the user not exercising, the current body water amount of the user based on Equation 2 below. Here, d may represent the fourth weight value, e may represent the fifth weight value, andfmay represent the sixth weight value. Further, n1 may represent the first coefficient, n4 may represent a fourth coefficient, and n5 may represent a fifth coefficient:

$$Body water amount of use who is not exercising = d \times third sensing {data}^{n 1} + e \times fourth sensing {data}^{n 4} + f \times fifth sensing {data}^{n 5} \qquad ( Equation 2 )$$

At this time, a which is the first weight value and d which is the fourth weight value may be set as a same value. However, b which is the second weight value and e which is the fifth weight value may be set to different values from each other, and may have different signs from each other. In addition, c which is the third weight value and f which is the sixth weight value may be set to different values from each other, and may have different signs from each other. Here, the first to sixth weight values and the first to fifth coefficients may be experimentally set.

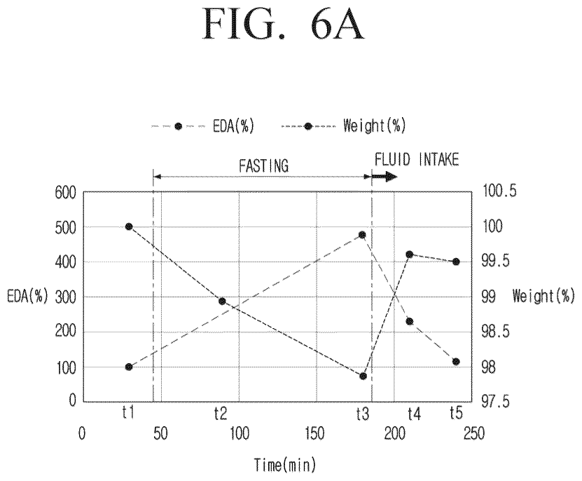

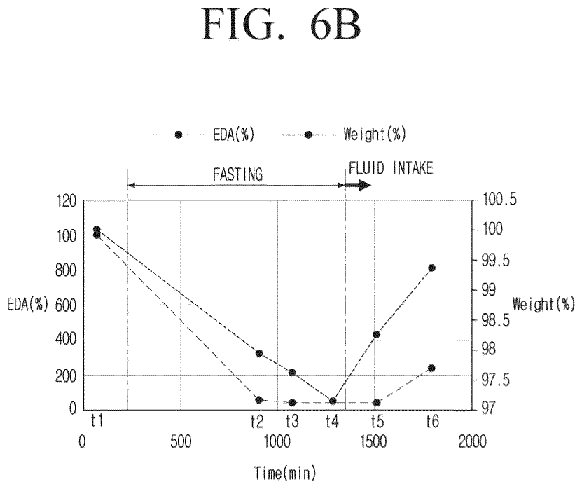

FIG. 6A and FIG. 6B are diagrams each illustrating changes in sensing data obtained by an EDA sensor based on user dehydration while a user is exercising and while the user is not exercising.

Referring to FIG. 6A, if the user is exercising, the sensing data obtained by the EDA sensor (i.e., fifth sensing data) may be increased according to the progression of user dehydration. Then, after a t6 time-point at which the user took in fluids, sensing data obtained by the EDA sensor decreased. Conversely, referring to FIG. 6B, if the user is not exercising (e.g., if the user has not taken in fluids for a long time), sensing data obtained by the EDA sensor (i.e., fifth sensing data) may be decreased according to the progression of user dehydration. Then, after the t6 time-point at which the user took in fluids, sensing data (i.e., fifth sensing data) obtained by the EDA sensor decreased. In other words, despite the same dehydration symptoms occurring, the change in fifth sensing data obtained by the EDA sensor may vary according to whether the user is exercising or whether the user is not exercising. Accordingly, the processor 130 may obtain the current body water amount of the user by applying coefficients of different signs from each another with respect to the sensing data obtained by the same sensor according to whether the user is exercising or the user is not exercising.

Meanwhile, the processor 130 according to one or more embodiments of the disclosure may obtain, based on the user exercising, the current body water amount of the user based on Equation 3 below. Here, g may represent a seventh weight value, h may represent an eighth weight value, and i may represent a ninth weight value. Further, n6 may represent a sixth coefficient, n7 may represent a seventh coefficient, and n8 may represent an eighth coefficient. In addition, Δδ1 may represent an amount of change of the obtained third sensing data relative to a reference third sensing data, Δδ2 may represent an amount of change of the obtained fourth sensing data relative to a reference fourth sensing data, and Δδ3 may represent an amount of change of the obtained fifth sensing data relative to a reference fifth sensing data:

$$Body water amount of use who is exercising = g \times Δδ {1}^{n 6} + h \times Δ {δ2}^{n 7} + i \times Δ {δ3}^{n 8} \qquad ( Equation 3 )$$

In addition, the processor 130 may obtain, based on the user not exercising, the current body water amount of the user based on Equation 4 below. Here, j may represent a tenth weight value, k may represent an eleventh weight value, and l may represent a twelfth weight value. Then, n6 may represent the sixth coefficient, n9 may represent a ninth coefficient, and n10 may represent a tenth coefficient. In addition, Δδ1 may represent the amount of change of the obtained third sensing data relative to the reference third sensing data, Δδ2 may represent the amount of change of the obtained fourth sensing data relative to the reference fourth sensing data, and Δδ3 may represent the amount of change of the obtained fifth sensing data relative to the reference fifth sensing data:

$$Body water amount of use who is not exercising = j \times Δ {δ1}^{n 6} + k \times Δ {δ2}^{n 9} + l \times Δ δ {3}^{n 10} \qquad ( Equation 4 )$$

At this time, g which is the seventh weight value and j which is the tenth weight value may be set as the same value. However, h which is the eighth weight value and k which is the eleventh weight value may be set to different values from each other, and may have different signs from each other. In addition, i which is the ninth weight value and 1 which is the twelfth weight value may be set to different values from each other, and may have different signs from each other. Here, the seventh to twelfth weight values and the fifth to tenth coefficients may be experimentally set.

Meanwhile, according to one or more embodiments of the disclosure, the processor 130 may identify a body water change rate of the user based on a previous body water amount of the user obtained from a previous period and the current body water amount of the user. At this time, the processor 130 may predict, based on the body water change rate of the user being greater than or equal to a threshold value, dehydration to occur to the user and provide information guiding a fluid intake recommendation through the output interface 120.

Specifically, the processor 130 may identify the body water amount of the user based on second sensing data that was periodically obtained. The processor 130 may calculate the body water change rate of the user based on the body water amount of the user identified from the previous period and the currently identified body water amount of the user. Then, the processor 130 may identify, based on the body water change rate being greater than or equal to a threshold value, a decrease in body water of the user as significant and predict that dehydration is to occur to the user. Accordingly, the processor 130 may provide guide information recommending fluid intake to the user.

Specifically, the processor 130 may predict, based on the body water change rate being greater than or equal to the threshold value even before the current body water amount of the user has reached the reserve reference body water amount, dehydration to occur to the user and output information guiding a fluid intake recommendation through the output interface 120. For example, the processor 130 may obtain, based on the user being identified as exercising, second sensing data about the user through the second sensor for every ten minutes which is the first period. Then, the processor 130 may identify, based on the obtained second sensing data, the body water amount of the user for every ten minutes (first period). The processor 130 may repeatedly identify whether the body water amount of the user has not yet reached the reference body water amount based on the identified body water amount (or repeatedly identify whether the body water amount of the user has reached the reserve reference body water amount), and store information about the body water amount in the memory 140.

At this time, the processor 130 may identify the body water change rate of the user even if a current body water amount, which is identified by obtaining the second sensing data at 3:10 pm which is current time, has not reached 54.4 kg which is the reference body water amount and 54.56 kg which is the reserve reference body water amount. Specifically, the processor 130 may identify the body water change rate of the user based on the body water amount of the user obtained ten minutes (first period) before (i.e., obtained at 3:00 pm) and the current body water amount. At this time, the identified body water change rate of the user (more accurately, an absolute value of the body water change rate) may be greater than or equal to the threshold value, and the processor 130 may predict dehydration symptoms to occur to the user by determining that the body water of the user is likely to reduce significantly. Accordingly, the processor 130 may provide guide information recommending fluid intake to the user through the output interface 120.

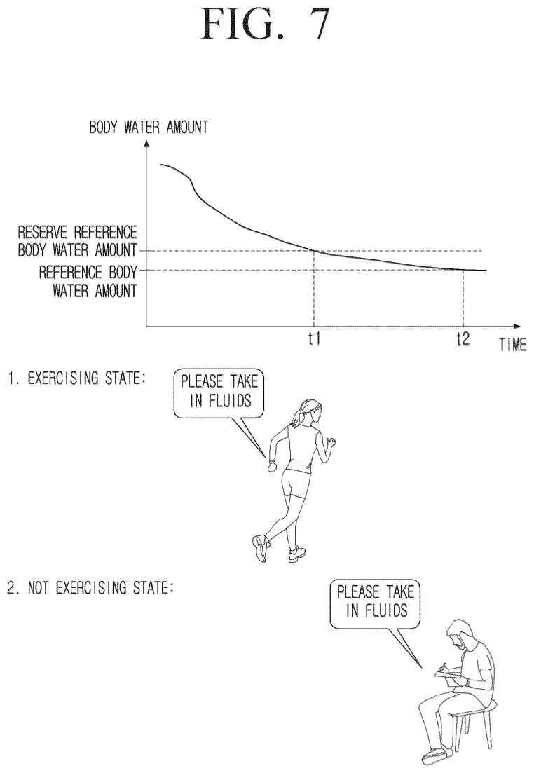

FIG. 7 is a diagram illustrating an example of outputting guide information recommending fluid intake to a user who is exercising and a user who is not exercising at different time-points according to one or more embodiments of the disclosure.

Referring to FIG. 7, the processor 130 may identify, based on the user exercising, whether the body water amount of the user has not yet reached the reference body water amount based on the reserve reference body water amount. Conversely, the processor 130 may identify, based on the user not exercising, whether the body water amount of the user has not yet reached the reference body water amount based on the reserve reference body water amount. Accordingly, if in the state of exercise to which the reserve reference body water amount having a relatively higher value than the reference body water amount is applied, guide information may be output earlier than in the state of not exercising. In other words, if the user is exercising, guide information may be output at the t1 time-point whereas, if the user is not exercising, guide information may be output at a t2 time-point which is later than the t1 time-point.

Meanwhile, the processor 130 may determine whether the body water amount of the user has not yet reached the reference body water amount or has reached the reference body water amount without having to calculate the current body water amount of the user. Specifically, the processor 130 may set each of the sensing data as dehydration factors, and determine whether current body water amount of the user has not yet reached the reference body water amount or has reached the reference body water amount by determining an amount of change of each of the dehydration factors (i.e., sensing data). One or more embodiments of the disclosure for predicting or determining dehydration symptoms of users based on dehydration factors will be described in detail below.

The processor 130 according to one or more embodiments of the disclosure may further include the memory 140 stored with a plurality of first reference values corresponding to when exercising and a plurality of second reference values corresponding to when not exercising.

Specifically, the memory 140 may be stored with the plurality of first reference values which are used in determining whether the body water amount of the user has not yet reached the reference body water amount. Here, the plurality of first reference values may correspond to sensing data obtained by the impedance sensor, sensing data obtained through the PPG sensor, and sensing data obtained through the EDA sensor, respectively. The sensing data obtained by the impedance sensor may be referred to as the sixth sensing data, the sensing data obtained through the PPG sensor may be referred to as the seventh sensing data, and the sensing data obtained through the EDA sensor may be referred to as the eighth sensing data below. In other words, the plurality of first reference values may include a reference value (a first-1 reference value) corresponding to the sixth sensing data obtained by the impedance sensor, a reference value (a first-2 reference value) corresponding to the seventh sensing data obtained through the PPG sensor, and a reference value (a first-3 reference value) corresponding to the eighth sensing data obtained by the EDA sensor. However, the embodiment is not limited thereto, and the plurality of first reference values may include the sensor used to identify the body water amount of the user, and various number of reference values according to the sensing data.

In addition, the memory 140 may be stored with the plurality of second reference values used in determining whether the body water amount of the user has reached the reference body water amount. Here, the plurality of second reference values (a second-1 reference value, a second-2 reference value, and a second-3 reference value) may correspond to the sixth sensing data obtained by the impedance sensor, the seventh sensing data obtained through the PPG sensor, and the eighth sensing data obtained through the EDA sensor, respectively.

Meanwhile, the plurality of first reference values and the plurality of second reference values may be set based on the reference body water amount, respectively. For example, the plurality of second reference values may be set respectively based on the sixth sensing data, the seventh sensing data, and the eighth sensing data obtained when the body water amount of the user is identified as having been reduced by 2%.

Then, the plurality of first reference values may be set respectively by applying a pre-set percentage to each of the sixth sensing data, the seventh sensing data, and the eighth sensing data obtained when the body water amount of the user is identified as reduced by 2% of the body weight of the user. At this time, the percentages applied to each of the sensing data (i.e., the sixth sensing data, the seventh sensing data, and the eighth sensing data) may be different, and may be set experimentally, respectively.

In addition, the plurality of first reference values may be set based on the reserve reference body water amount. For example, if the reserve reference body water amount is a value identified as body water of 1.8% body weight of the user as being lost, the plurality of second reference values may be set respectively by applying the pre-set percentage to each of the sixth sensing data, the seventh sensing data, and the eighth sensing data obtained when the body water amount of the user is identified as having reduced by 1.8% of the body weight of the user.

In addition, the processor 130 may set the plurality of first and second reference values based on sensing data obtained from the previous period. In an example, the processor 130 may set, based on the sixth, seventh, and eighth sensing data obtained from the previous period, the first reference values based on the values of the sixth, seventh, and eighth sensing data obtained when the reserve reference body water amount is reached in the following period. In addition, the processor 130 may set, based on the sixth, seventh, and eighth sensing data obtained from the previous period, the second reference values based on the values of the sixth, seventh, and eighth sensing data obtained when the reference body water amount is reached in the following period.

The processor 130 may compare, based on the user being identified as exercising, each of the plurality of second sensing data obtained through the one or more sensors 110 according to the first body water analysis algorithm with the plurality of first reference values. Then, the processor 130 may provide information guiding a fluid intake recommendation before the current body water amount of the user reaches the reference body water amount based on a first comparison result through the output interface 120.

Specifically, the processor 130 may compare the sixth sensing data, the seventh sensing data, and the eighth sensing data obtained through the plurality of sensors based on the first body water analysis algorithm with the plurality of first reference values. Here, the first body water analysis algorithm may be associated with a criterion for determining a subject of comparison for each of the sensing data (sixth sensing data, seventh sensing data, and eighth sensing data) from among the plurality of first reference values, a method for comparing each of the sensing data with each of the subjects for comparison, weight values applied to each of the sensing data and reference values, a margin of error that is applied to comparison results, a method for interpreting the comparison results, and the like.

Specifically, the processor 130 may obtain, based on the user being identified as exercising, a change rate relative to each of the first reference values by comparing the first reference values corresponding to each of the sixth sensing data, the seventh sensing data, and the eighth sensing data based on the first body water analysis algorithm, and obtain the first comparison result based on the change rates relative to each of the first reference values.

In other words, the processor 130 may obtain the change rate of the sixth sensing data obtained based on the impedance sensor relative to the first-1 reference value, obtain the change rate of the seventh sensing data obtained based on the PPG sensor relative to the first-2 reference value, and obtain the change rate of the eighth sensing data obtained based on the EDA sensor relative to the first-3 reference value.

At this time, according to one or more embodiments of the disclosure, the processor 130 may identify whether a plurality of change rates from among the change rates corresponding to each of the sixth sensing data, the seventh sensing data, and the eighth sensing data satisfies a pre-set first change condition. Then, the processor 130 may provide, based on the pre-set first change condition being identified as satisfied, information guiding a fluid intake recommendation before the current body water amount of the user reaches the reference body water amount through the output interface 120. As such, the field of dehydration monitoring is further improved by such features by providing advance indications to a user of possibly impending dehydration even before a set dehydration threshold may be reached.

Specifically, the processor 130 may identify whether a change rate corresponding to the sixth sensing data satisfies a pre-set first-1 condition. Specifically, the processor 130 may identify whether the change rate corresponding to the sixth sensing data is increased by a pre-set first size (or percentage). Then, the processor 130 may identify whether a change rate corresponding to the seventh sensing data satisfies a pre-set first-2 condition. Specifically, the processor 130 may identify whether the change rate corresponding to the seventh sensing data is reduced by a pre-set second size (or second percentage). Then, the processor 130 may identify whether a change rate corresponding to the eighth sensing data satisfies a pre-set first-3 condition. Specifically, the processor 130 may identify whether the change rate corresponding to the eighth sensing data is increased by a pre-set third size (or third percentage). In other words, the processor 130 may identify whether the body water amount of the user has not yet reached the pre-set body water amount by determining whether there is a decrease in the seventh sensing data obtained by the PPG sensor, and determining whether there is an increase in the sixth and eighth sensing data obtained by the impedance sensor and the EDA sensor, respectively.

At this time, the processor 130 may identify as the body water amount of the user not yet reaching the pre-set body water amount when at least two or more conditions from among the above-described first-1, first-2, and first-3 conditions are satisfied.

In addition, the processor 130 may compare, based on the user being identified as not exercising, each of the plurality of second sensing data obtained through the one or more sensors 110 according to the second body water analysis algorithm with the plurality of second reference values. Then, the processor 130 may provide information guiding a fluid intake recommendation when the current body water amount of the user reaches the reference body water amount based on a second comparison result through the output interface 120.

Specifically, the processor 130 may compare the sixth sensing data, the seventh sensing data, and the eighth sensing data obtained through the plurality of second sensors based on the second body water analysis algorithm with the plurality of second reference values. Here, the second body water analysis algorithm may be associated with a criterion for determining a subject of comparison for each of the sensing data (sixth sensing data, seventh sensing data, and eighth sensing data) from among the plurality of first reference values, a method for comparing each of the sensing data with each of the subjects for comparison, weight values applied to each of the sensing data and reference values, a margin of error that is applied to comparison results, a method for interpreting the comparison results, and the like.

Specifically, if the user is identified as exercising, the change rates relative to each of the second reference values may be obtained by comparing the second reference values corresponding to each of the sixth sensing data, the seventh sensing data, and the eighth sensing data, and the second comparison result may be obtained based on the change rates relative to each of the second reference values.

In other words, the processor 130 may obtain a change rate of the sixth sensing data obtained based on the impedance sensor relative to the second-1 reference value, obtain a change rates of the seventh sensing data obtained based on the PPG sensor relative to the second-2 reference value, and obtain a change rates of the eighth sensing data obtained based on the EDA sensor relative to the second-3 reference value.

At this time, according to one or more embodiments of the disclosure, the processor 130 may provide information guiding a fluid intake recommendation through the output interface 120 when a plurality of change rates from among the change rates corresponding to each of the sixth sensing data, the seventh sensing data, and the eighth sensing data satisfies a pre-set second change condition as and when the current body water amount of the user reaches the reference body water amount.

Specifically, the processor 130 may identify whether the change rate corresponding to the sixth sensing data satisfies a pre-set second-1 condition. Specifically, the processor 130 may identify whether the change rate corresponding to the sixth sensing data is increased by the pre-set first size (or percentage). At this time, a first dehydration factor (i.e., sixth sensing data) from among a plurality of dehydration factors determined while in a state of not exercising may be same in condition as when in a state of exercising.

Then, the processor 130 may identify whether the change rate corresponding to the seventh sensing data satisfies a pre-set second-2 condition. Specifically, the processor 130 may identify whether the change rate corresponding to the seventh sensing data is increased by a pre-set fourth size (or fourth percentage). Then, the processor 130 may identify whether the change rate corresponding to the eighth sensing data satisfies a pre-set second-3 condition. Specifically, the processor 130 may identify whether the change rate corresponding to the eighth sensing data is reduced by a pre-set fifth size (or fifth percentage). In other words, the processor 130 may identify whether the body water amount of the user has not yet reached the pre-set body water amount by determining whether there is a decrease in the eighth sensing data obtained by the PPG sensor, and by determining whether there is an increase in the sixth and seventh sensing data obtained by each of the impedance sensor and the EDA sensor.

In addition, the processor 130 may determine, based on the user being in a state of not exercising unlike the state of exercising, whether the change rate of the seventh sensing data is increased, and determine whether the change rate of the eighth sensing data is decreased.

At this time, the processor 130 may identify as the body water amount of the user not yet reaching the pre-set body water amount when at least two or more conditions from among the above-described second-1, second-2, and second-3 conditions are satisfied.

An embodiment of the disclosure of the processor 130 outputting guide information respectively to a user who is exercising or a user who is not exercising will be described below.

According to one or more embodiments of the disclosure, the processor 130 may output, based on the user who is exercising being identified as dehydrated, first guide information including at least one from among a message recommending to adjust the intensity of exercise to the user and a message recommending fluid intake through the output interface 120.

Then, the processor 130 may output, based on the user who is not exercising being identified as dehydrated, second guide information including at least one from among a message warning of danger of chronic dehydration to the user and a message recommending to improve living habits of the user through the output interface 120.

In other words, the processor 130 may output guide information suitable to each state of the user through the output interface 120. Specifically, if dehydration symptoms are identified as having occurred to the user who is exercising, the processor 130 may output a message recommending to the user who is exercising to take in fluids such as beverages, and the like. In addition, the processor 130 may output, according to the change in reduction of body water of the user, a message warning of dehydration symptoms occurring and a message recommending to adjust the exercise intensity when the user proceeds to continue with the exercise.

Meanwhile, the processor 130 may output, based on the user who is not exercising being identified as dehydration symptoms having occurred, guide information including a message recommending to improve living habits of the user and a message warning of chronic dehydration danger of the user. Specifically, because the reason for dehydration symptoms occurring with users who are not exercising can be related with the living habits of the user in daily life, the processor 130 may provide a message to improve the living habits or to warn of chronic dehydration symptoms to the user.

Meanwhile, the processor 130 may accumulate and identify a number of times the current body water amount of the user who is not exercising has reached the reference body water amount. At this time, if a number of accumulated times relative to a base date exceeds a pre-set number of times, the processor 130 may output the number of times the body water amount reached the reference body water amount together with a message recommending to improve living habits to the user. For example, if the reference date is one day, and the pre-set number of times is two times, the processor 130 may identify a number of times the current body water amount of the user reached the reference body water amount for one day. At this time, if the identified number of times relative to one day exceeds two times, the processor 130 may output guide information including a message recommending to improve living habits to the user and a message warning of chronic dehydration danger of the user.

FIG. 8 is a diagram illustrating a detailed configuration of the electronic apparatus 100 according to one or more embodiments of the disclosure.

Referring to FIG. 8, the electronic apparatus 100 may include the one or more sensors 110, the output interface 120, a speaker 121, a display 122, the memory 140, a communication interface 150, a user interface 160. Detailed descriptions of overlapped configurations shown in FIG. 2 from among the configurations of the electronic apparatus 100 shown in FIG. 8 will be omitted.

The speaker 121 may output guide information recommending fluid intake in voice message form. To this end, the speaker 121 may be formed of a tweeter for playing high-range sound, a midrange for playing mid-range sound, a woofer for playing low-range sound, a sub-woofer for playing ultra-low range sound, an enclosure for controlling resonance, a cross-over network dividing electric signal frequencies which are input to the speaker 121 into bandwidths, and the like.

The speaker 121 may output sound signals to outside of the electronic apparatus 100. The speaker 121 may output playing of multi-media, playing of recordings, various notification sounds, voice messages, and the like. The electronic apparatus 100 may include audio output devices such as the speaker 121, but may include an output device such as an audio output terminal. Specifically, the speaker 121 may provide obtained information, information processed and/or manufactured based on the obtained information, a response result or operation result for a user voice, and the like in a voice form.

The display 122 may output various visual information. Specifically, the display 122 may display guide information recommending fluid intake in message form. To this end, the display 122 may be implemented as a display 122 including self-emissive devices or, as a display 122 including non-emissive devices and a backlight. For example, the above may be implemented as displays 122 of various forms such as, for example, and without limitation, a liquid crystal display (LCD), an organic light emitting diode (OLED) display 122, light emitting diodes (LED), a micro LED, a mini LED, a plasma display panel (PDP), a quantum dot (QD) display 122, a quantum dot light-emitting diodes (QLED), or the like. In the display 122, a driving circuit, which may be implemented in the form of an a-si TFT, a low temperature poly silicon (LTPS) TFT, an organic TFT (OTFT), or the like, a backlight unit, and the like may be included.

Meanwhile, the display 122 may be implemented as a touch screen coupled with a touch sensor, a flexible display, a rollable display, a 3D display, a display physically connected with a plurality of display modules, and the like.

Meanwhile, the display 122 may function as the user interface 160 that performs an interacting function through a touch input of the user when implemented as the touch screen.

The processor 130 may control the display 122 to output an output image obtained according to the various embodiments described above. Here, the output image may be a high-resolution image of 4K or greater than or equal to 8K.

The memory 140 may store data necessary for various embodiments. For example, the memory 140 may be stored with information about the body water amount of the user which is periodically identified, and stored with information about the reference body water amount (and the reserve reference body water amount), the plurality of first reference values, and the plurality of second reference values. The memory 140 may be implemented in a form of memory 140 embedded in the electronic apparatus 100 according to a data storage use, or implemented in a form of memory 140 attachable to or detachable from the electronic apparatus 100. For example, data for the driving of the electronic apparatus 100 may be stored in the memory 140 embedded in the electronic apparatus 100, and data for an expansion function of the electronic apparatus 100 may be stored in the memory 140 attachable to or detachable from the electronic apparatus 100.

Meanwhile, the memory 140 embedded in the electronic apparatus 100 may be implemented as at least one of a volatile memory (e.g., a dynamic RAM (DRAM), a static RAM (SRAM), or a synchronous dynamic RAM (SDRAM)), or a non-volatile memory (e.g., a one time programmable ROM (OTPROM), a programmable ROM (PROM), an erasable and programmable ROM (EPROM), an electrically erasable and programmable ROM (EEPROM), a mask ROM, a flash ROM, a flash memory (e.g., NAND flash or NOR flash), a hard disk drive, or a solid state drive (SSD)). In addition, the memory 140 attachable to or detachable from the electronic apparatus 100 may be implemented in a form such as, for example, and without limitation, a memory card (e.g., a compact flash (CF), a secure digital (SD), a micro secure digital (micro-SD), a mini secure digital (mini-SD), an extreme digital (xD), a multi-media card (MMC), etc.), an external memory (e.g., a USB memory) connectable to a USB port, or the like.

The communication interface 150 may input and output data of various types. For example, the communication interface 150 may transmit and receive data of various types with an external device (e.g., a source device), an external storage medium (e.g., USB memory 140), or an external server (e.g., WEBHARD) through communication methods such as, for example, and without limitation, an AP based Wi-Fi (wireless LAN network), Bluetooth, ZigBee, a wired/wireless local area network (LAN), a wide area network (WAN), Ethernet, IEEE 1394, a high-definition multimedia interface (HDMI), a universal serial bus (USB), a mobile high-definition link (MHL), Audio Engineering Society/European Broadcasting Union (AES/EBU), Optical, Coaxial, or the like.

According to an example, the communication interface 150 may include a Bluetooth Low Energy (BLE) module. The BLE may refer to Bluetooth technology capable of transmitting and receiving low-power and low-capacity data in a 2.4 GHz frequency bandwidth having a reach radius of approximately 10 m. However, the above is not limited thereto, and the communication interface 150 may include a Wi-Fi communication module. In other words, the communication interface 150 may include at least one from among the BLE module or the Wi-Fi communication module.

The user interface 160 may be a configuration for the electronic apparatus 100 to perform an interaction with the user. For example, the user interface 160 may include at least one from among a touch sensor, a motion sensor, a button, a jog dial, a switch, and a microphone, but is not limited thereto.

Meanwhile, methods according to the various embodiments of the disclosure described above may be implemented in an application form installable in electronic apparatuses 100 of the related art. Alternatively, the methods according to the various embodiments of the disclosure described above may be performed using a deep learning based artificial neural network (or deep artificial neural network), that is, a trained network model.

In addition, the methods according to the various embodiments of the disclosure described above may be implemented with only a software upgrade, or a hardware upgrade for the electronic apparatuses of the related art.

In addition, the various embodiments of the disclosure described above may be performed through an embedded server provided in the electronic apparatus, or an external server of the electronic apparatus.

Meanwhile, according to one or more embodiments of the disclosure, the various embodiments described above may be implemented with software including instructions stored in a machine-readable storage media (e.g., computer). The machine may call stored instructions from a storage medium, and as an apparatus operable according to the called instructions, may include an electronic apparatus (e.g., electronic apparatus (A)) according to the above-mentioned embodiments. Based on a command being executed by the processor, the processor may directly or using other elements under the control of the processor perform a function corresponding to the command. The command may include a code generated by a compiler or executed by an interpreter. A machine-readable storage medium may be provided in a form of a non-transitory storage medium. Herein, ‘non-transitory’ merely means that the storage medium is tangible and does not include a signal, and the term does not differentiate data being semi-permanently stored or being temporarily stored in the storage medium.

In addition, according to one or more embodiments of the disclosure, the method according to the various embodiments described above may be provided included a computer program product. The computer program product may be exchanged between a seller and a purchaser as a commodity. The computer program product may be distributed in a form of the machine-readable storage medium (e.g., a compact disc read only memory (CD-ROM)), or distributed online through an application store (e.g., PLAYSTORE™). In the case of online distribution, at least a portion of the computer program product may be stored at least temporarily in the storage medium such as a server of a manufacturer, a server of an application store, or a memory of a relay server, or temporarily generated.

In addition, each of the elements (e.g., a module or a program) according to various embodiments described above may be configured as a single entity or a plurality of entities, and a portion of sub-elements of the above-mentioned relevant sub-elements may be omitted, or other sub-elements may be further included in the various embodiments. Alternatively or additionally, a portion of the elements (e.g., modules or programs) may be integrated into one entity to perform the same or similar functions performed by the respective relevant elements prior to integration. Operations performed by a module, a program, or another element, in accordance with various embodiments, may be executed sequentially, in a parallel, repetitively, or in a heuristic manner, or at least a portion of the operations may be executed in a different order, omitted or a different operation may be added.

While the disclosure has been illustrated and described with reference to example embodiments thereof, it will be understood that the embodiments are intended to be illustrative, not limiting. It will be understood by those skilled in the art that various changes in form and details may be made therein without departing from the true spirit and full scope of the disclosure, including the appended claims and their equivalents.

What is claimed is:

1. An electronic apparatus, comprising:
one or more sensors;
an output interface; and
one or more processors configured to:
identify, based on first sensing data obtained through the one or more sensors sensing at least any of acceleration of a user, electrical activity of a skin of the user, and a blood volume of the skin of the user, whether the user is exercising;
determine, based on the user being identified as exercising and on second sensing data obtained through the one or more sensors and a first body water analysis algorithm, whether a current body water amount of the user reaches a reference body water amount;
provide, through the output interface and based on the user being identified as exercising, first information guiding a fluid intake recommendation before the current body water amount of the user is determined to reach the reference body water amount;
determine, based on the user being identified as not exercising and on the second sensing data and a second body water analysis algorithm that is different from the first body water analysis algorithm, whether the current body water amount of the user reaches the reference body water amount; and
provide, through the output interface and based on the user being identified as not exercising, second information guiding the fluid intake recommendation when the current body water amount of the user is determined to reach the reference body water amount.

2. The electronic apparatus of claim 1,
wherein the one or more processors are further configured to:
obtain, based on the user being identified as exercising, the current body water amount of the user by applying the first body water analysis algorithm to the second sensing data obtained through the one or more sensors, and
obtain, based on the user being identified as not exercising, the current body water amount of the user by applying the second body water analysis algorithm to the second sensing data obtained through the one or more sensors.

3. The electronic apparatus of claim 2,
wherein the one or more sensors comprise an impedance sensor, a Photoplethysmogram (PPG) sensor, and an Electro Dermal Activity (EDA) sensor,
wherein the second sensing data comprises third sensing data, fourth sensing data, and fifth sensing data obtained respectively through the impedance sensor, the PPG sensor, and the EDA sensor such that the impedance sensor obtains the third sensing data, the PPG sensor obtains the fifth sensing data, and the EDA sensor obtains the fifth sensing data,
wherein the one or more processors are further configured to:
obtain, based on the user being identified as exercising, a body water amount of the user by applying a first weight value, a second weight value, and a third weight value to the third sensing data, the fourth sensing data, and the fifth sensing data, respectively, and
obtain, based on the user being identified as not exercising, the body water amount of the user by applying a fourth weight value, a fifth weight value, and a sixth weight value to the third sensing data, the fourth sensing data, and the fifth sensing data, respectively,
wherein the first weight value is same as the fourth weight value,
wherein the second weight value is different in sign from the fifth weight value such that a first one of the second weight value and the fifth weight value is positive and a second one of the second weight value and the fifth weight value is negative, and
wherein the third weight value is different in sign from the sixth weight value.

4. The electronic apparatus of claim 2,
wherein the one or more processors are further configured to:
obtain, based on the user being identified as exercising, the second sensing data at each of a plurality of first periods through the one or more sensors; and
obtain, based on the user being identified as not exercising, the second sensing data at each of a plurality of second periods through the one or more sensors, and
wherein each one of the plurality of first periods is shorter in time than each one of the plurality of second periods.

5. The electronic apparatus of claim 4,
wherein the one or more processors are further configured to:
identify, based on the user being identified as not dehydrated, a body water change rate of the user based on a previous body water amount of the user obtained from a previous period, of any of the plurality of first periods and the plurality of second periods, and a current body water amount of the user; and
provide, based on the body water change rate of the user being greater than or equal to a threshold value, information predicting that dehydration is to occur and guiding the fluid intake recommendation to the user through the output interface.

6. The electronic apparatus of claim 1, further comprising:
a memory storing a plurality of first reference values, corresponding to when the user is to be identified as exercising, and a plurality of second reference values corresponding to when the user is to be identified as not exercising,
wherein the one or more processors are further configured to:
compare, based on the user being identified as exercising, each of a plurality of second sensing data obtained through the one or more sensors according to the first body water analysis algorithm and the plurality of first reference values;
provide, through the output interface and based on the user being identified as exercising, the first information as guiding the fluid intake recommendation before the current body water amount of the user reaches the reference body water amount based on a first comparison result;
compare, based on the user being identified as not exercising, each of the plurality of second sensing data obtained through the one or more sensors according to the second body water analysis algorithm and the plurality of second reference values; and
provide, through the output interface and based on the user being identified as not exercising, information guiding the fluid intake recommendation when the current body water amount of the user reaches the reference body water amount based on a second comparison result.

7. The electronic apparatus of claim 6,
wherein the one or more sensors comprise an impedance sensor, a Photoplethysmogram (PPG) sensor, and an Electro Dermal Activity (EDA) sensor,
wherein the second sensing data comprises sixth sensing data, seventh sensing data, and eighth sensing data obtained respectively through the impedance sensor, the PPG sensor, and the EDA sensor such that the impedance sensor obtains the sixth sensing data, the PPG sensor obtains the seventh sensing data, and the EDA sensor obtains the eighth sensing data, and
wherein the one or more processors are further configured to:
compare, based on the user being identified as exercising, a first reference value corresponding to each of the sixth sensing data, the seventh sensing data, and the eighth sensing data and obtain first change rates relative to first reference values of each thereof, and obtain the first comparison result based on the first change rates relative to first reference values of each thereof; and
compare, based on the user being identified as exercising, a second reference value corresponding to each of the sixth sensing data, the seventh sensing data, and the eighth sensing data and obtain second change rates relative to second reference values of each thereof, and obtain the second comparison result based on the second change rates relative to second reference values of each thereof.

8. The electronic apparatus of claim 7,
wherein the one or more processors are further configured to:
provide, through the output interface and based on the user being identified as exercising, the first information as guiding the fluid intake recommendation before the current body water amount of the user reaches the reference body water amount and based on determining that a plurality of the first change rates, from among the first change rates, corresponding to each of the sixth sensing data, the seventh sensing data, and the eighth sensing data satisfies a pre-set first change condition, and
provide, through the output interface and based on the user being identified as not exercising, the second information as guiding the fluid intake recommendation when the current body water amount of the user is determined to reach the reference body water amount and based on a plurality of the second change rates, from among the second change rates, corresponding to each of the sixth sensing data, the seventh sensing data, and the eighth sensing data satisfies a pre-set second change condition.

9. The electronic apparatus of claim 1,
wherein the one or more processors are further configured to:
obtain, based on the user being identified as exercising, biometric data of the user through the one or more sensors for a pre-set time; and
re-identify whether the user is exercising by comparing the biometric data and with reference biometric data of the user, and
wherein the biometric data of the user comprises one or more of a heart rate, a temperature, and skin electrical signals.

10. The electronic apparatus of claim 1,
wherein the one or more processors are further configured to:
output, through the output interface and based on the user being identified both as exercising and as dehydrated, first guide information comprising at least one from among a first message, recommending to the user of adjusting an exercise intensity, and a second message recommending an intake of a beverage; and
output, through the output interface and based on the user being identified both as not exercising and as dehydrated, second guide information comprising at least one from among a third message, warning of a danger of chronic dehydration to the user, and a fourth message recommending to improve living habits of the user.

11. The electronic apparatus of claim 1, wherein
the electronic apparatus is a smart watch configured to be worn by the user,
the smart watch is configured to, while worn by the user, automatically and in real-time:
control the one or more sensors to obtain the first sensing data;
determine, based on the first sensing data, whether the user is exercising;
determine, based on the user being identified as exercising and on the second sensing data and the first body water analysis algorithm, whether the current body water amount of the user reaches the reference body water amount;
provide, through the output interface and based on the user being identified as not exercising, the first information,
determine, based on the user being identified as not exercising and on the second sensing data and the second body water analysis algorithm that is different from the first body water analysis algorithm, whether the current body water amount of the user reaches the reference body water amount; and
providing, through the output interface and based on the user being identified as not exercising, the second information.

12. A method for controlling an electronic apparatus, the method, by one or more processors of the electronic apparatus, comprising:
identifying, based on first sensing data obtained through one or more sensors of the electronic apparatus sensing at least any of acceleration of a user, electrical activity of a skin of the user, and a blood volume of the skin of the user, whether the user is exercising;
determining, based on the user being identified as exercising and on second sensing data obtained through the one or more sensors and a first body water analysis algorithm, whether a current body water amount of the user reaches a reference body water amount;
providing, through an output interface of the electronic apparatus and based on the user being identified as exercising, first information guiding a fluid intake recommendation before the current body water amount of the user is determined to reach the reference body water amount;
determining, based on the user being identified as not exercising and on the second sensing data and a second body water analysis algorithm that is different from the first body water analysis algorithm; and
providing, through the output interface and based on the user being identified as not exercising, second information guiding the fluid intake recommendation when the current body water amount of the user is determined to reach the reference body water amount.

13. The method of claim 12,
wherein providing the first guide information comprises obtaining, based on the user being identified as exercising, the current body water amount of the user by applying the first body water analysis algorithm to the second sensing data obtained through one or more sensors, and
wherein providing the second guide information comprises obtaining, based on the user being identified as not exercising, the current body water amount of the user by applying the second body water analysis algorithm to the second sensing data obtained through the one or more sensors.

14. The method of claim 13,
wherein the one or more sensors comprise an impedance sensor, a Photoplethysmogram (PPG) sensor, and an Electro Dermal Activity (EDA) sensor,
wherein the second sensing data comprises third sensing data, fourth sensing data, and fifth sensing data obtained respectively through the impedance sensor, the PPG sensor, and the EDA sensor such that the impedance sensor obtains the third sensing data, the PPG sensor obtains the fifth sensing data, and the EDA sensor obtains the fifth sensing data,
wherein the providing first guide information comprises obtaining, based on the user being identified as exercising, a body water amount of the user by applying a first weight value, a second weight value, and a third weight value to the third sensing data, the fourth sensing data, and the fifth sensing data, respectively, and
wherein the providing second guide information comprises obtaining, based on the user being identified as not exercising, the body water amount of the user by applying a fourth weight value, a fifth weight value, and a sixth weight value to the third sensing data, the fourth sensing data, and the fifth sensing data, respectively,
wherein the first weight value is same as the fourth weight value,
wherein the second weight value is different in sign from the fifth weight value such that a first one of the second weight value and the fifth weight value is positive and a second one of the second weight value and the fifth weight value is negative, and
wherein the third weight value is different in sign from the sixth weight value.

15. The method of claim 13,
wherein providing the first guide information comprises obtaining, based on the user being identified as exercising, the second sensing data at each of a plurality of first periods through the one or more sensors,
wherein providing the second guide information comprises obtaining, based on the user being identified as not exercising, the second sensing data at each of a plurality of second periods through the one or more sensors, and
wherein the each one of the plurality of first periods is shorter in time than each one of the plurality of second periods.

16. A non-transitory computer-readable recording medium storing computer instructions for an electronic apparatus to perform operations that, when executed by one or more processors of the electronic apparatus, cause the electronic apparatus to implement:
identifying, based on first sensing data obtained through one or more sensors of the electronic apparatus sensing at least any of acceleration of a user, electrical activity of a skin of the user, and a blood volume of the skin of the user, whether the user is exercising;
determining, based on the user being identified as exercising and on second sensing data obtained through the one or more sensors and a first body water analysis algorithm, whether a current body water amount of the user reaches a reference body water amount;
providing, through an output interface of the electronic apparatus and based on the user being identified as exercising, first information guiding a fluid intake recommendation before the current body water amount of the user is determined to reach the reference body water amount;
determining, based on the user being identified as not exercising and on the second sensing data and a second body water analysis algorithm that is different from the first body water analysis algorithm; and
providing, through the output interface and based on the user being identified as not exercising, second information guiding the fluid intake recommendation when the current body water amount of the user is determined to reach the reference body water amount.
